(19) United States
(12) Patent Application Publication
(10) Pub. No.: US 2024/0376506 A1
(43) Pub. Date: Nov. 14, 2024

(54) PROCESS FOR BIOCONJUGATE PRODUCTION

(71) Applicants: JANSSEN PHARMACEUTICALS, INC., Titusville, NJ (US); GLAXOSMITHKLINE BIOLOGICALS S.A., Rixensart (BE)

(72) Inventors: Helen Heide Erik THOMAS, Bern (CH); Marc Sylvain NOVERRAZ, Bern (CH); Matthias Dominik KAESER, Bern (CH); Caspar Ludwig Hermann SCHÄFER, Bern (CH); Martin Maximilian SCHEIDT, Bern (CH); Denis Louis Raymond BASCOUL, Bern (CH); Maximilian BREUER, Bern (CH); Martin Edward BRAUN, Schlieren (CH); Gilles-Antoine MARTIN, Schlieren (CH); Markus Daniel MÜLLER, Schlieren (CH); Dominique Nicolas SIRENA, Schlieren (CN)

(21) Appl. No.: 18/285,088

(22) PCT Filed: Apr. 7, 2022
(86) PCT No.: PCT/EP2022/059323

§ 371 Date: Sep. 29, 2023

(30) Foreign Application Priority Data
Apr. 8, 2021 (EP) 21167452.8

(51) Int. Cl. C12P 19/04; C07K 1/18; C07K 1/20; C07K 1/34; C08B 37/00; C12P 21/00

(52) U.S. Cl. CPC C12P 19/04; C07K 1/18; C07K 1/20; C07K 1/34; C08B 37/0003; C12P 21/00

(57) ABSTRACT

The present invention relates to a process for the production of purified O-polysaccharide-Exoprotein A (O-EPA) bioconjugates. In particular, the process comprises a purification of said bioconjugate comprising the steps of a first anion exchange chromatography prior to a hydroxyapatite chromatography followed by a hydrophobic interaction chromatography and subsequently a second anion exchange chromatography.

### CROSS-REFERENCE TO RELATED APPLICATIONS

This application is a Section 371 of International Application No. PCT/EP2022/059323 filed Apr. 7, 2022, which was published under International Publication No. WO 2022/214620 A1, which claims priority to European patent application 21167452.8, filed Apr. 8, 2021, the disclosures of which are incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “P181588PCOO_seq_list” and a creation date of Aug. 23, 2023 and having a size of 6 KB. The sequence listing submitted via EFS-Web is part of the specification and is herein incorporated by reference in its entirety.

### BACKGROUND OF THE INVENTION

There is an increased pressure of regulatory authorities on biopharmaceutical manufacturers to provide glycoconjugate-based drugs with high purity. However, the purification of complex glycoconjugates, such as bioconjugates comprising an O-polysaccharide covalently linked to a carrier protein, is a challenging task. Such bioconjugates are a key component of glycoconjugate vaccine compositions, such as a multivalent vaccine composition currently under development for the prevention of infectious diseases caused by “extraintestinal pathogenic Escherichia coli (ExPEC)” strains [Poolman and Wacker, J. Infect. Dis. (2016) v. 213(1):6-13].

Such bioconjugates can generally be produced by enzymatic conjugation of the O-polysaccharide (O-PS) component to a carrier protein in prokaryotic host cells, such as E. coli, using e.g. the PglB oligosaccharyltransferase system [see e.g. WO 2015/124769; WO 2020/191082; Poolman and Wacker, J. Infect. Dis. (2016) v. 213(1), pp. 6-13 and references therein]. In this method, coupling of the O-PS component to the carrier protein occurs in the periplasmic space, i.e. the space between the inner cytoplasmic membrane and the outer membrane of Gram-negative bacteria, such as E. coli.

The production, including the purification of such bioconjugates, is known per se.

### BRIEF SUMMARY OF THE INVENTION

The present invention relates to a process for the production of purified O-polysaccharide-Exoprotein A (O-EPA) bioconjugates. In particular, the process comprises a purification of said bioconjugate comprising the steps of a first anion exchange chromatography prior to a hydroxyapatite chromatography followed by a hydrophobic interaction chromatography and subsequently a second anion exchange chromatography.

Previously described processes, in particular the corresponding purification steps, while giving acceptable purity and being suitable on relatively smaller scale could be improved with the aim of efficient large-scale manufacturing of such bioconjugates (e.g. culturing of the host cell culture in bioreactors with a volume of at least 100 L up to 20,000 L) that would be needed to obtain sufficient amounts of safe product for vaccination of a large number of people in an economically feasible manner, i.e. having an acceptable overall yield and very high purity of the final product. Each purification step will need to be selected, specifically combined, and subsequently optimized to yield bioconjugates that are suitable as a component of glycoconjugate vaccines, particularly multivalent ExPEC vaccines. Such bioconjugates must be provided with high purity and manufactured at a commercial scale, e.g. in bioreactors with a volume of between 100 L to 20,000 L. In view of all the factors that can vary, finding optimal conditions for producing glycoconjugate vaccines against ExPEC is burdened with challenges, and the choice of purification steps as well as the order of the respective steps, is a priori unpredictable, which applies a fortiori for a full process that yields various different glycoconjugates at very high purity and good yields at commercial scale.

Burckhardt et al. [Vaccine (2019), 37(38):5762-5769] describe a purification process for the production of a non-conjugated EPA carrier protein from whole cell lysates. The aim of the described purification process is to reduce the host cell protein (HCP) content compared to previously used purification methods for said EPA carrier protein. The described process involves an anionic exchange chromatography step (AEX; Capto Q resin) followed by purification on two mixed mode resins (MEP HyperCell and subsequent hydroxyapatite resin) instead of a previously used hydrophobic interaction chromatography step (HIC; Phenyl sepharose resin) in combination with a second AEX step (AEX2, Q Sepharose resin) and size exclusion chromatography [SEC; Qian et al., Vaccine (2007), 25(20):3923-3933]. However, the substrate of the purification process, i.e. a non-conjugated EPA carrier protein, differs significantly from EPA conjugates, such as O-EPA bioconjugates (e.g. for the O-EPA conjugates that are the interest of the present invention the actual most relevant active moieties are the O-antigen polysaccharides, that are intended to generate serotype-specific immune responses when used in a vaccine composition). Further, whole cell lysates used as a starting material for the described purification process differ from the periplasmic fraction, such as the osmotic shock fraction that is used for the purification of O-EPA bioconjugates. Accordingly, the problem to be solved by this method is not applicable to the aim of the inventive production process described herein because both, the substrate and the matrix from which it is purified are different.

Ravenscroft et al. [Glycobiology (2016), 26(1):51-62] used the PglB conjugation system described above for the production of a glycoconjugate comprising an EPA carrier protein and a Shigella dysenteriae type 1 (Sd1) O-polysaccharide (O-PS). Isolation of the Sd1 O-EPA conjugate included its extraction from the periplasmic space by osmotic shock and purification using a combination of AEX and SEC. In more detail: In a first step, the osmotic shock fraction was purified on a Source 15Q resin, i.e. a first AEX step (AEX1). Sd1 O-EPA containing fractions were identified by SDS PAGE, pooled, concentrated, and, upon buffer exchange by tangential flow filtration (TFF), re-applied on the Source 15Q resin (AEX2). As a third step, the O-EPA conjugate was purified by SEC on a Superdex 200 column. However, whereas the described purification process is suitable for the small to medium scale production of bioconjugates, i.e incubation of the host cell culture in a bioreactor containing up to 50 L incubation medium, it requires further improvement for large scale production in an economically feasible manner, e.g. incubation of the host cell culture in a bioreactor containing at least 100 L and up to 20,000 L incubation medium, e.g. from 150 L to 5000 L, especially if the product is intended to be a mixture of various glycoconjugates that need to be suitable as pharmaceutical components, i.e. can be safely injected into mammals, in particular into humans.

Van den Dobbelsteen et al. [Vaccine (2016), 34:4152-4160] describe a study investigating the immunogenicity and safety of a tetravalent E. coli O-antigen bioconjugate vaccine in animal models. The biomass for obtaining the bioconjugates was produced by incubation of the host cell culture in a 10 L working volume bioreactor. For studies in rabbits and mice, bioconjugates were released from the periplasm by osmotic shock and purified over two anion exchange chromatography steps.

For studies in rats, bioconjugates were released using high-pressure homogenisation and purified using a pre-purification step on an anion exchange chromatography column, a capture step on an anion exchange chromatography column, a hydrophobic interaction chromatography step, a polishing step on an anion exchange chromatography column and a final polishing step on a size-exclusion chromatography column.

WO 2017/067964 describes the use of PcrV as a new carrier protein from Pseudomonas aeruginosa and conjugates comprising an antigen covalently linked to PcrV carrier protein. Purification of an EPA-06 bioconjugate is described using Metal-chelate affinity chromatography (IMAC), AEX and SEC.

WO 2009/104074 describes a production process for O-EPA bioconjugates, in particular Shigella O1-EPA bioconjugates. Specifically, E. coli host cells comprising genetic information encoding PglB, EPA and the enzymes for the biosynthesis of the Shigella O1 polysaccharide, were cultured in a 2 L bioreactor. Periplasmic proteins were extracted by osmotic shock and O1-EPA bioconjugates were purified from the periplasmic fraction from 100,000 OD of cells. Purification was performed by two consecutive AEX steps (AEX1 and AEX2; Source Q resin), followed by SEC (Superdex 200 Hi Load 26/60 resin). In an additional embodiment, Shigella O1-EPA bioconjugates were purified using a combination of AEX and fluoroapatite chromatography. Again, although suitable for small to medium-scale production processes (e.g. in bioreactors with a volume of up to 50 L), the described purification procedure can be improved for the reproducible large scale manufacture of O-EPA bioconjugates with the aim to obtain a safe product for vaccination of a large number of people.

WO 2017/035181 describes multivalent vaccines containing conjugates of E. coli polysaccharide antigens O25B, O1A, 02, and 06A each covalently bound to EPA carrier protein and uses of the vaccine to provide immune protection against ExPEC infection. Conjugates were purified using two consecutive AEX steps and an SEC step.

WO 2014/057109 describes methods for inserting nucleic acid sequences into host cells and the production of glycoconjugate vaccine candidates at 10 L scale in a bioreactor. Conjugates were purified using a first AEX step, a second AEX step and SEC.

WO 2015/124769 describes a production process for bioconjugates for the development of a tetravalent ExPEC vaccine, said bioconjugates comprising an EPA carrier protein with in particular 2 or 4 glycosylation sites and a PS component corresponding to E. coli O-antigens, in particular O25A or O25B. Bioconjugates produced from 50 L of culture medium were purified by two consecutive AEX steps, followed by SEC. In another example, bioconjugates obtained from 2 L of culture medium were purified by IMAC. Again, although suitable for the purification of bioconjugates from up to 50 L of culture medium, as required for instance for pre-clinical experiments, the described production process requires further improvement for the large-scale manufacture of O-EPA bioconjugates with high purity as is required for providing a multivalent ExPEC vaccine on a commercial scale.

Although previously described processes are suitable for small to medium-scale production (e.g. in bioreactors with a volume of up to 50 L), there is a need for an improved process for the production of bioconjugates, in particular E. coli O-antigen-EPA conjugates, that is amenable to large scale manufacturing.

Thus, it is an object of the present invention to mitigate this drawback of the state of the art and to provide such a process.

In particular, it is an aim of the present invention to provide a process that is amenable to the large scale production of bioconjugates in bioreactors, such as bioreactors with a volume of 100 L up to 20,000 L, e.g. with a volume of between 150 L and 5000 L.

Further it is an aim of the present invention to provide a process for the production of bioconjugates which requires a minimum of purification steps in order to allow the cost-efficient production of such bioconjugates.

Further, it is an aim of the present invention to provide a process for the production of bioconjugates that may be adapted to meet the purity requirements of different applications, such as for applications in the veterinary field or in humans.

One or more of the above objectives are achieved by a process for the production of bioconjugates as defined in claim 1. Further aspects of the invention are disclosed in the specification and independent claims, preferred embodiments are disclosed in the specification and the dependent claims.

The present invention will be described in more detail below. It is understood that the various embodiments, preferences and ranges as provided/disclosed in this specification may be combined at will. Further, depending on the specific embodiment, selected definitions, embodiments or ranges may not apply.

Unless otherwise stated, the following definitions shall apply in this specification:

As used herein, the term “a”, “an”, “the” and similar terms used in the context of the present invention (especially in the context of the claims) are to be construed to cover both the singular and plural unless otherwise indicated herein or clearly contradicted by the context.

As used herein, the terms “including”, “containing” and “comprising” are used herein in their open, non-limiting sense. It is understood that the various embodiments, preferences and ranges may be combined at will.

As used herein, the term “about,” when used in conjunction with a number, refers to any number within ±1, ±5 or ±10% of the referenced number.

Throughout this specification a number of abbreviations are used, including:
AEX1 a first Anion EXchange chromatography
AEX2 a second Anion EXchange chromatography
cHA ceramic hydroxyapatite
CV column volume
EPA exoprotein A (also referred to as exotoxin A) of Pseudomonas aeruginosa
FPF Filtered Periplasmic host cell Fraction
HA Hydroxyapatite
HCP Host Cell Proteins
HIC Hydrophobic Interaction Chromatography
HPLC High-Performance Liquid Chromatography
IMAC Immobilized Metal Affinity Chromatography
O-EPA glycoconjugate of EPA carrier protein covalently coupled to bacterial O-antigen polysaccharide
SEC Size Exclusion Chromatography
SE-HPLC Size Exclusion—High-Performance Liquid Chromatography
TFF Tangential Flow Filtration
WFI water for injection

The term “glycoconjugate” is known in the field and particularly describes chemical entities covalently bound to one or more polysaccharide(s). Such glycoconjugate may be obtained by biological conjugation in a living cell (“bioconjugate” or “biological conjugate”) or may be obtained by chemical conjugation of a polysaccharide (“chemical” or “synthetic” glycoconjugate). Particularly suitable chemical entities are proteins, the corresponding glycoconjugates being glycoproteins. Specifically, the term glycoconjugates relates to a conjugation product wherein a polysaccharide (i.e. a glycan) is covalently coupled to a carrier protein.

The term glycoprotein includes “traditional glycoproteins” and “glycoconjugate vaccines”. In traditional glycoproteins, the emphasis is on the protein part, such as for instance for antibodies or erythropoietin where the ‘active’ principle is more residing in the protein part, and the glycans play a role for instance in half-life or defining other properties. Such traditional glycoproteins find widespread use in pharmaceutical applications. In glycoconjugate vaccines, the emphasis is on the glycan part, to which an immune response is desired because the glycans are the relevant antigens, and the protein part merely serves as a carrier to lead to a desired T-cell memory immune response.

The term “polysaccharide” is known in the field and particularly describes polymeric carbohydrates composed of monosaccharide units bound together by glycosidic linkages, either linear or branched. Such polysaccharides are characterized by their repeating units, each repeating unit described with their respective monosaccharide composition. Said repeating units include one or more monosaccharides which can also be chemically modified (e.g. amidated, sulphonated, acetylated, phosphorylated, etc). Typically found monosaccharides in said repeating units are cyclic or linear monosaccharides containing three to seven carbon atoms. In the specific case of glycoconjugate vaccines, the conjugated polysaccharide originates from a pathogenic species (e.g. Escherichia coli) with said repeating unit defined by the genetics of the specific pathogen. The repeating unit can thus be a specific marker/identifier of the pathogen.

The term “polysaccharide component” consequently denotes one or more glycan chain(s) of a glycoconjugate. Glycans can be monomers or polymers of sugar residues, but typically contain at least three sugars, and can be linear or branched. A glycan may include natural sugar residues (e.g., glucose, N-acetylglucosamine, N-acetyl neuraminic acid, galactose, mannose, fucose, arabinose, ribose, xylose, etc.) and/or modified sugars (e.g., 2′-fluororibose, 2′-deoxyribose, phosphomannose, 6′-sulfo N-acetylglucosamine, etc). The term “glycan” includes homo- and heteropolymers of sugar residues. The term “glycan” also encompasses a glycan component of a glycoconjugate (e.g., of a glycoprotein, glycopeptide).

The term “O-acetylated polysaccharide”, as used herein, refers to polysaccharides where one or more monosaccharides of the repeating unit are chemically modified by acetylation. Said monosaccharides have one or more of their present hydroxyl groups acetylated. For pathogen-derived repeating units used in glycoconjugate vaccines, the O-acetylation of certain monosaccharides can be essential to induce an immune response for said pathogen. Examples of pathogen-derived polysaccharide components are shown in Table 1.

The terms “glycan”/“glycan chain” are synonyms of “polysaccharide” as defined below. Correspondingly, in the context of this invention, “glycan” and the prefix “glyco-”, also refers to the carbohydrate portion of a glycoconjugate, such as a glycoprotein.

The term “serotype” as used herein, refers to glycoconjugates having different polysaccharide chains which are derived from different bacterial serotypes. Examples of glycans from a number of E. coli serotypes are identified below in Table 1.

The term “adjusting a load” as used herein, refers to adjusting a load of a process intermediate, e.g. the periplasmic fraction of a host cell comprising an O-EPA conjugate or the O-EPA comprising fraction after a first, second or third purification step, to conditions that are suitable for applying said process intermediate on a chromatography resin for further purification. Unless specified otherwise or clearly contradicted by the context, “adjusting a load” refers to adjusting the conductivity of a load to a target conductivity that is suitable for the following purification step and adjusting the pH of a load to a target pH that is suitable for the following purification step. Further, “adjusting a load” includes adjusting the concentration of a process intermediate, i.e. reducing the processing volume, particularly by TFF.

The terms “resin” and “medium” are used herein synonymously and relate to a chromatography resin or a chromatography medium that is used for separation of a target protein(s), i.e. the O-EPA bioconjugate, from impurities.

Resins useful for the invention can be in different formats, e.g. as beads, filters (membranes), cartridges, etc., all to be considered as ‘resin’ according to the invention. In certain embodiments the resins are in the form of beads that can be used in columns. In certain embodiments the resins are in the form of membranes with functional groups. In certain embodiments the resins are in the form of directly usable cartridges. Resins that can be used according to the invention can be commercially obtained from vendors, e.g. Cytiva (former GE Healthcare), Bio-Rad, and/or others.

The term “drug substance” as used herein refers to the bulk product of an individual bioconjugate (e.g. E. coli O-antigen polysaccharide covalently coupled to EPA carrier protein, e.g. E. coli O25B O-antigen covalently coupled to EPA), that is at higher concentration than the product as will be finally administered to a subject in need thereof. The drug substance can be produced after purification of the bioconjugate. The drug substance can, for example, be stored in a more concentrated form in a suitable formulation buffer (see e.g. WO 2018/077853), for instance in frozen condition, e.g. at −70° C.

The term “drug product” as used herein refers to the formulation of the bioconjugates particularly E. coli O-antigen polysaccharides individually coupled to EPA carrier protein, in final form for administration to a subject in need thereof. As used herein, the term “drug product” particularly relates to a multivalent vaccine composition, e.g. a four-valent ExPEC glycoconjugate vaccine composition comprising the E. coli O-antigen polysaccharides O25B, O1A, O2 and O6A, each individually coupled to an EPA carrier protein. Further non-limiting examples of multivalent glycoconjugate vaccine compositions are e.g. a nine-valent glycoconjugate vaccine compositions comprising the E. coli O-antigen polysaccharides O1A, O2, O4, O6A, O15, O16, O18A, O25B and O75, and e.g. a ten-valent glycoconjugate vaccine composition comprising the E. coli O-antigen polysaccharides O1A, O2, O4, O6A, O8, O15, O16, O18A, O25B and O75, each E. coli O-antigen polysaccharide being individually coupled to an EPA carrier protein. Drug product can typically be prepared by mixing the drug substances of the respective glycoconjugates, and dilution by a suitable formulation buffer if needed (see e.g. WO 2018/077853), such that the target dose of vaccine is produced.

### BRIEF DESCRIPTION OF THE DRAWINGS

The present invention will be better understood by reference to the figures.

FIG. 1: Fractions of AEX1 eluate analyzed by SDS-PAGE and Coomassie staining. The gel shows from left to right Molecular weight marker (1), Standard of purified O25B-EPA bioconjugate from reference batch (2), Load material of the same process stage of a reference batch (3), Load (4), Flow through (5), Wash (6), fraction 1 (7), fraction 2 (8), fraction 3 (9), fraction 4 (10). The majority of the O-EPA bioconjugate eluted in fractions 1 and 2 (lanes 7 and 8), whereas in fractions 3 and 4 (lanes 9 and 10) the relative amount of impurities was increased.

FIG. 2A and FIG. 2B: Fractions of cHA eluate analyzed by SDS-PAGE and Coomassie staining. FIG. 2A) Gel 1 showing from left to right: Molecular weight marker (1), Standard of purified O25B-EPA bioconjugate from reference batch (2), Load material of the same process stage of a reference batch (3), Load (4), Flow through (5), Wash (6), Fraction 1-3 (7), Fraction 4 (8), Fraction 5 (9), Fraction 6 (10). FIG. 2B) Gel 2 showing from left to right: Standard of purified O25B-EPA bioconjugate from reference batch (11), Molecular weight marker (12), Fraction 7 (13), Fraction 8 (14), Fraction 9 (15), Fraction 10 (16), Fraction 11 (17), Fraction 12-14 (18). Elution of O-EPA bioconjugate is visible starting from fraction 7 (13). Starting from fraction 11, the amount of O-EPA bioconjugate decreases whereas product is still visible but in decreasing amounts, impurities are more abundant.

FIG. 3: Fractions of HIC eluate analyzed by SDS-PAGE and Coomassie staining. The gels show from left to right: Molecular weight marker (1), Standard of purified O25B-EPA bioconjugate from reference batch (2), Load material of the same process stage of a reference batch (3), Load (4), Flow through (5), Wash (6), Fraction 1 (7), Fraction 2 (8), Fraction 3 (9), Fraction 4 (10).

FIGS. 4A-4D: Fractions of AEX2 eluate analyzed by SDS-PAGE and Coomassie staining. FIG. 4A) Gel 1 showing from left to right: Molecular weight marker (1), Standard of purified O25B-EPA bioconjugate from reference batch (2), Load material of the same process stage of a reference batch (3), Load (4), Flow through (5), Wash (6), empty (7), Fraction 1 (8), Fraction 2 (9), Fraction 3 (10). FIG. 4B) Gel 2 showing from left to right: Standard of purified O25B-EPA bioconjugate from reference batch (11), Molecular weight marker (12), Fraction 4 (13), Fraction 5 (14), Fraction 6 (15), Fraction 7 (16), Fraction 8 (17), Fraction 9 (18), Fraction 10 (19), Fraction 11 (20). FIG. 4C) Gel 3 showing from left to right: Molecular weight marker (21), Standard of purified O25B-EPA bioconjugate from reference batch (22), Fraction 12 (23), Fraction 13 (24), Fraction 14 (25), Fraction 15 (26), Fraction 16 (27), Fraction 17 (28), Fraction 18 (29), Molecular weight marker (30). FIG. 4D) Gel 4 showing from left to right: Standard of purified O25B-EPA bioconjugate from reference batch (31), Molecular weight marker (32), Fraction 19 (33), Fraction 20 (34), strip (35), empty lanes (36-39), standard of purified O25B-EPO bioconjugate from reference batch (40). In fractions 1-4, mainly transaldolase (impurity) is eluting in decreasing amounts. Starting from fraction 5, the O25B-EPA bioconjugate is visible, with high intensity starting from fraction 6.

FIG. 5: Purity of glycosylated EPA as measured by SE-HPLC throughout the production process. Intermediates measured after Capture AEX (1), cHA (2), HIC (3) and Polishing AEX (4) steps. Purity in the end of this example is 99.7%. In general, after HIC, a purity between 92-100% can be reached, after Polishing AEX, a purity between 93-100% can be reached.

### DETAILED DESCRIPTION OF THE INVENTION

In more general terms, in a first aspect the invention relates to a process for the production of a purified O-polysaccharide-ExoProtein A carrier (O-EPA) conjugate. Said process comprises providing the O-EPA conjugate as a bioconjugate obtained from prokaryotic host cells as well as a purification that comprises several chromatographic steps (herein described as steps b to e). This is explained in more detail below.

In a first step, the O-EPA bioconjugate is provided as a periplasmic fraction of prokaryotic host cells that express said bioconjugate (step a).

In a second step the O-EPA bioconjugate is purified by a first anion exchange chromatography (AEX1; step b). In particular, the AEX1 step is a capture step. In certain embodiments, the AEX1 is performed with a strong anion exchange resin. In certain embodiments, the resin is a ceramic resin, e.g. having rigid, non-compressible characteristics, and has high dynamic binding capacities at high flow rates. A non-limiting example of a resin that is particularly suitable for the AEX1 step is a Q Ceramic HyperD F resin. In preferred embodiments, the AEX1-step is performed in a bind-elute mode.

In a third step, the O-EPA conjugate is further purified by hydroxyapatite (HA) chromatography (step c). In preferred embodiments, the HA comprises ceramic hydroxyapatite (cHA). Ceramic HA is a spherical ceramic form of crystalline hydroxyapatite and this form allows it to be used in production-scale columns at high flow rates while maintaining its unique separation properties. HA chromatography is sometimes described as a mixed-mode (or multi-modal) form of chromatography. In preferred embodiments, the HA, particularly cHA, chromatography is performed in bind-elute mode. The third step is performed after the second step.

In a preferred embodiment, the O-EPA conjugate is in a fourth step further purified by hydrophobic interaction chromatography (HIC; step d) and in a fifth step further purified by a second anion exchange chromatography (AEX2; step e). This embodiment is the most preferred, and outlined in example 1. This embodiment gives the highest purity of the O-EPA conjugate as compared to other embodiments indicated below with less chromatography steps, while still being efficient enough in that the overall yield is acceptable for a large scale process for making pharmaceutical preparations of bioconjugates (e.g. at least 5% overall yield from a bioreactor of at least 150 L, preferably at least 10% overall yield, e.g. about 5-30% overall yield [relative to O-EPA conjugate present in a filtered periplasmic fraction], with a purity of at least 95%).

In certain embodiments, the HIC step is performed using a hydrophobic interaction medium. A non-limiting example of a hydrophobic interaction medium that can be used for HIC of the invention is a Sartobind Phenyl absorber.

In certain embodiments, the AEX2 step is performed using a strong anion exchange resin. A non-limiting example of an anion exchange medium that can be used for AEX2 of the invention is a Source Q resin.

Preferably, the inventive process does not comprise an SEC step. Although SEC can be conveniently used in small to medium scale production processes (i.e. incubation of the host cell culture in a bioreactor containing up to 50 L incubation medium), scalability of SEC is limited. Hence, a process that does not include an SEC step has the advantage of being well suited for economic large scale manufacturing of glycoconjugates (i.e. incubation of the host cell culture in a bioreactor containing at least 100 L and up to 20,000 L incubation medium, e.g. from 150 L to 5000 L).

However, as illustrated by its frequent use in small to medium scale production processes, developing a process for providing high purity glycoconjugates for human use that does not include an SEC step is particularly challenging.

Preferably, the inventive process does not comprise additional chromatography steps. Thus, the inventive process preferably does not include more than 4 chromatography steps. Although addition of further chromatography steps is a common strategy to increase the purity of the obtained product, this will generally lead to lower overall yields and render the process more laborious. Moreover, addition of further chromatography steps is generally associated with an increase in solvent consumption. Thus, a process including not more than 4 chromatography steps is preferred for production of glycoconjugates at commercial scale, both for economic and ecological reasons.

Preferably, the inventive process is performed at large scale. Thus the inventive process preferably includes incubation of a prokaryotic host cell in a bioreactor with a volume of between 100 L and 20000 L, preferably with a volume of between 150 L and 5000 L, e.g. 150 L-1000 L, such as 200 L.

In another embodiment, the O-EPA conjugate is in a fourth step further purified by hydrophobic interaction chromatography (HIC; step d) and no further chromatographic step is applied.

In another embodiment, the O-EPA conjugate is in a fourth step further purified by a second anion exchange chromatography (AEX2; step e) and no further chromatographic step is applied. This embodiment is outlined in example 2. This is preferred over the previous embodiment (i.e. omitting HIC instead of AEX2 is preferred from practical process perspective, although both embodiments result in similar purity of the O-EPA bioconjugate).

In another embodiment, the O-EPA conjugate is in a fourth step further purified by a second anion exchange chromatography (AEX2; step e) and in a fifth step further purified by hydrophobic interaction chromatography (HIC; step d).

In general, in ion exchange chromatography, binding is based on electrostatic charges. In case of AEX, the resin has positively charged functional groups, thus sample components with negatively charged functional groups will bind to it. AEX can be performed using weak anion exchangers or strong anion exchangers, as known to the skilled person. In certain embodiments of the invention, strong anion exchange resins are used for the AEX steps (AEX1 and AEX2). Non-limiting examples of functional groups that are suitable for AEX resins are quaternary ammonium groups. Such resins are commercially available and include e.g. a Q Ceramic HyperD F resin and a Source 15Q resin. Based upon the present disclosure and common knowledge, the skilled person will know how to vary different available AEX resins for use in a process of the invention. As one non-limiting example AEX1 can suitably be performed with a Q Ceramic HyperD F resin and AEX2 can suitably be performed with a Source 15Q resin. With increasing salt concentration, salt ions in the elution buffer compete for binding with the resin-bound material, and the bound material is displaced and eluted. Alternatively, when pH is changed, bound proteins are titrated and eventually become non-charged or have the same charge as the functional groups of the resin, leading to repulsion and elution of the bound protein.

HA, particularly cHA chromatography, can be viewed as a mixed-mode (multimodal) chromatography step. Such resins are commercially available, e.g. a CHT Ceramic Hydroxyapatite Type 1 resin. In HA (or cHA), separation is in particular achieved by a combination of ionic and metal-affinity interactions. Typically, protein(s) bind to HA via HA-phosphoryl (cation-exchange) or HA-calcium interactions (metal affinity). Small basic proteins usually bind to HA by phosphoryl-cation exchange and acidic proteins typically interact predominantly by calcium affinity. However, large proteins often bind using both mechanisms [Saraswat et al., 2013, BioMed Research International, Article ID 312709].

During HIC, target protein(s), e.g. O-EPA bioconjugate, are separated from impurities based on their hydrophobicity. Target protein(s) containing hydrophobic and hydrophilic regions are typically applied to a HIC column in a high-salt buffer. The salt in the buffer reduces the solvation of target protein(s). As solvation decreases, hydrophobic regions that become exposed are adsorbed by the HIC resin. The more hydrophobic the molecule, the less salt is needed to promote binding. Usually a decreasing salt gradient is used to elute samples from the column in order of increasing hydrophobicity. Sample elution may also be assisted by the addition of further components, such as detergents, to the elution buffer. A non-limiting example of a HIC capsule that is suitable for performing the above HIC step is a Sartobind Phenyl Jumbo 5 L capsule. The skilled person is able to vary the HIC resin and use it according to the invention, based upon the present disclosure and the various commercially available HIC resins.

In preferred embodiments, AEX1 is performed in bind-elute mode.

In preferred embodiments, HA (cHA) is performed in bind-elute mode.

In preferred embodiments, HIC is performed in bind-elute mode.

In preferred embodiments, AEX2 is performed in bind-elute mode.

In preferred embodiments, AEX1, HA (cHA), HIC and AEX2 are performed in bind-elute mode.

In preferred embodiments, the AEX1 step is a capture step.

In preferred embodiments, the AEX2 step is a polishing step.

This aspect of the invention, particularly the process steps and terms used, are explained in further detail below:

Bioconjugate: The term is discussed above. Specifically, a bioconjugate is a glycoconjugate prepared in a host cell, wherein the host cell machinery produces the glycan and the carrier protein and links the glycan to the carrier protein, e.g., via N-links of asparagine or arginine. A particularly preferred host cell for producing bioconjugates is E. coli, preferably comprising nucleic acid encoding: (i) the carrier protein, (ii) an oligosaccharyltransferase such as C. jejuni PglB that is capable of covalently linking O-antigen polysaccharides to an asparagine (Asn) residue in a glycosylation consensus sequence (Asn-X-Ser(Thr), wherein X can be any amino acid except Pro) in a carrier protein via N-linked glycosylation, and (iii) an rfb gene cluster encoding the enzymes responsible for generating the O-antigen polysaccharide of a desired serotype. By creating host cells with a different rfb locus, different bioconjugates can be prepared, e.g. comprising O-antigen polysaccharides from different E. coli or Shigella serotypes. Culturing such host cells will produce the bioconjugates comprising the carrier protein to which the O-antigen encoded by the rfb locus is covalently attached, within the periplasm of the host cell. A more detailed description for production of bioconjugates in such host cells can for instance be found in WO 2009/104074, WO 2015/124769, WO 2017/035181, or WO 2020/191082. Optimized variants of the PglB oligosaccharyltransferase for production of bioconjugates of specific E. coli O-antigens has been described in WO 2020/191088. The present invention deals with novel and improved methods of purification of the produced bioconjugates from such host cells. The host cell for production of bioconjugates is typically a prokaryotic cell, preferably a bacterial cell, preferably a gram-negative bacterial cell, and in preferred embodiments the host cell is E. coli. The O-EPA bioconjugate is thus to be purified from E. coli host cell proteins. One example of a host cell protein is transaldolase, in particular E. coli transaldolase, and the processes described in the instant invention are capable of obtaining O-EPA preparations with very low amounts of host cell proteins, including very low amounts of transaldolase, which surprisingly appeared one of the most residual host cell proteins that was hard to remove from O-EPA bioconjugates. The host cells typically are engineered to express the bioconjugates in the periplasm, and hence a good starting point to purify the O-EPA bioconjugates is from the periplasmic fraction of the host cells, e.g. from gram-negative bacterial host cells, such as E. coli host cells. Particularly useful bioconjugates include carrier proteins to which one or more polysaccharides are attached. Such bioconjugates are for instance used as the active components of certain vaccines, which aim at inducing functional immune responses against the polysaccharides of the bioconjugates. In embodiments of the invention, said bioconjugate comprises one carrier protein and one or more polysaccharides covalently bound to said carrier protein, preferably 1 to 4 polysaccharides covalently bound to said carrier protein.

In embodiments of the invention, the bioconjugate is a conjugation product containing an E. coli O-antigen covalently bound to a carrier protein. In embodiments of the invention, the bioconjugate is a conjugation product containing a Shigella O-antigen covalently bound to a carrier protein. The term O-antigen is known in the field and used in its normal context, it is not to be confused with O-linked. In typical embodiments, the O-antigen is N-linked to the carrier protein. The term O-antigen generally refers to a repetitive glycan polymer contained within an LPS of a bacteria, such as E. coli. The O-antigen of E. coli is a polymer of immunogenic repeating oligosaccharides (typically 1-40 repeating units, e.g. 5-30 repeating units) and typically used for serotyping and glycoconjugate vaccine production.

Carrier Protein: A particularly suitable carrier protein in the context of the invention is detoxified Exotoxin A of Pseudomonas aeruginosa (EPA) (the terms Exotoxin A and ExoProtein A of P. aeruginosa, or EPA, are used interchangeably). In particular embodiments, a carrier protein is a detoxified Exotoxin A of P. aeruginosa. For EPA, various detoxified protein variants have been described in literature and could be used as carrier proteins. For example, detoxification can be achieved by mutating and deleting the catalytically essential residues L552V and ΔE553.

Preferably, the EPA carrier protein comprises 1 to 20, preferably 1 to 10, preferably 2 to 4, glycosylation sites.

In a particular embodiment, the EPA comprises four glycosylation sites. See for example WO 2015/124769, WO 2017/035181, or WO 2020/191082 for a description of examples of bioconjugation of E. coli O-antigen polysaccharides to EPA carrier protein, or for example WO 2009/104074 for a description of examples of bioconjugation of Shigella O-antigen polysaccharides to EPA carrier protein. In one particular non-limiting embodiment, the carrier protein of a bioconjugate according to the invention comprises SEQ ID NO: 1. In certain embodiments, during expression in the host cell the carrier protein comprises a signal sequence that targets the carrier protein to the periplasmic space. Various signal sequences can be used. In one non-limiting embodiment, the signal sequence comprises SEQ ID NO: 2. A signal sequence may be cleaved off after translocation of the protein to the periplasm and may thus no longer be present in the final carrier protein of a bioconjugate.

Polysaccharide: The term is discussed above. Suitable polysaccharides comprise 1 to 100, such as 1-50, 1-40, 1-30, 1-20, and 1-10, 3-50, 3-40, e.g. at least 5, such as 5-40, e.g. 7-30, e.g. 7 to 25, e.g. 5 to 20, e.g. 10-20, repeating units n. Such repeating units contain (i.e. comprise or consist of) (i) non-modified mono-saccharides and/or (ii) modified monosaccharides. The term “modified mono-saccharides” in non-limiting embodiments includes N-acetylation, O-acetylation, amidation and/or amination of mono-saccharides. Such modified mono-saccharides may comprise zero, one, or more modifications, for example zero, one, two or three of the above modifications, at the same mono-saccharide.

In particular embodiments, modified monosaccharides are O-acetylated and/or N-acetylated monosaccharides, specifically monosaccharides comprising one O-acetylation or N-acetylation.

In embodiments of the invention, suitable repeating units comprise mono-saccharides selected from the group consisting of Mannose, Rhamnose, Glucose, Fucose, Galactose, modified Mannose, modified Rhamnose, modified Glucose, modified Fucose, and modified Galactose.

In embodiments of the invention, the O-polysaccharide is specific to a Gram-negative bacterium selected from the list of Escherichia and Shigella, preferably E. coli.

Non-limiting and exemplary structures of E. coli O-antigen polysaccharides are shown below in Table 1. A single repeating unit for each E. coli O-antigen polysaccharide is shown. In this table, each n is independently an integer of 1 to 100, such as 1-50, 1-40, 1-30, 1-20, and 1-10, 3-50, 3-40, e.g. at least 5, such as 5-40, e.g. 7-30, e.g. 7 to 25, e.g. 5 to 20, e.g. 10-20, but in some instances can be 1-2. In certain preferred embodiments for bioconjugate compositions of E. coli O-antigen polysaccharides purified according to the methods of the invention, n is on average somewhere between 5-30, preferably 10-25, preferably 10-20.

TABLE 1
Examples of structures of E. coli O-antigen polysaccharides
E. coli O-antigen Polysaccharide
Structure of Repeating Unit
O4 antigen polysaccharide (O4)

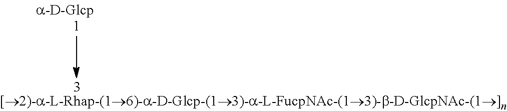

O1A antigen polysaccharide (O1A)

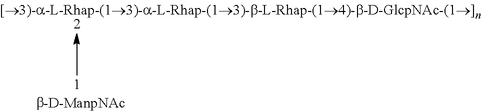

O2 antigen polysaccharide (O2)

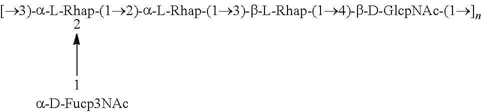

O6A antigen polysaccharide (O6)

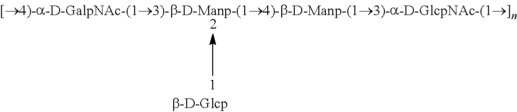

O8 antigen polysaccharide (08)
α-D-Manp3Me-(1→[3)-β-D-Manp-(1→2)-α-D-Manp-(1→2)-α-D-Manp-(1→]ₙ
O15 antigen polysaccharide (015)
[→2)-β-D-Galp-(1→3)-α-L-FucpNAc-(1→3)-β-D-GlcpNAc-(1→]ₙ
O16 antigen polysaccharide (O16)

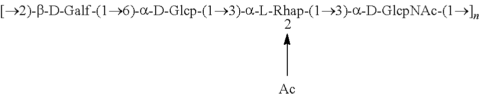

O18A antigen polysaccharide (O18A)

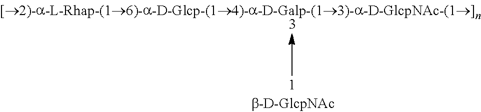

O25B antigen polysaccharide (O25B)

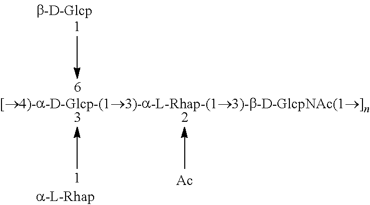

O75 antigen polysaccharide (O75)

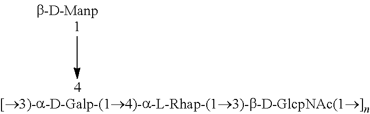

Structures of other E. coli or Shigella or other bacterial O-antigen polysaccharides from various serotypes are known and can be found in the art.

Capture step: The term is known in the field and relates to a first chromatography step with the objective to bind the protein(s) of interest from the crude sample and to isolate them from critical contaminants such as proteases and glycosidases. The target protein(s), i.e. the O-EPA bioconjugate(s), are concentrated and transferred to a buffer that will maintain the functional and structural integrity of the O-EPA bioconjugate. Removal of other critical contaminants may also be achieved by careful optimization of binding conditions.

The focus in optimizing a capture step is on capacity and speed. It may thus be acceptable to compromise on resolution in order to maximize the capacity and/or speed of the separation in this first step.

Polishing step: The term is known in the field and relates to a chromatography step that is performed as final chromatography step with the objective to further enhance the purity of the target protein, i.e. the O-EPA bioconjugate.

Bind-elute mode: The term is known in the field and relates to a mode of separation that works by first binding sample components, in particular the protein/bioconjugate of interest, to the chromatography resin. Once sample components are bound, the resin is washed with a buffer, and thus non-bound material is removed. Then, the bound material is eluted. This mode of separation is in contrast to the flow-through mode wherein the pH/ionic strength of the sample and buffer is selected in such a way that the protein will not bind but will flow through the column, leaving most or specific impurities bound.

In embodiments of the invention, in purification steps b) to e) conditions are first adjusted to allow binding of the O-EPA conjugate to a chromatography medium and subsequently adjusted to allow elution of the O-EPA conjugate from said medium, i.e. the chromatography step is performed in bind-elute mode.

Mixed-mode chromatography: The term is known in the field and used herein synonymously to multimodal chromatography. The term relates to chromatographic methods that utilize more than one form of interaction between the stationary phase and the target protein, i.e. the bioconjugate, in order to achieve separation from impurities. Mixed-mode resin selectivity typically results from a combination of electrostatic interactions, hydrophobic interactions and/or hydrogen bonding, depending on a case-to-case basis. In the case of HA or cHA chromatography, which is sometimes also viewed as a particular form of mixed-mode chromatography, separation is in particular achieved by a combination of ionic and metal-affinity interactions. Such resins are commercially available, e.g. a CHT Ceramic Hydroxyapatite XT resin.

Viable and non-viable particles: Viable particles are particles that contain one or more living microorganisms, such as bacteria. These can affect the sterility of the pharmaceutical product.

Non-viable particles are particles that do not contain living microorganisms but may act as transportation vehicles for viable particles.

Viable and non-viable particles generally range from about 0.2 μm to 30 μm in size, typically from about 0.2 μm to 5 μm.

The individual production steps outlined below are known per se when looked at individually. However, it was found that the specific combination of steps a-e described below is particularly suitable for the production of a variety of different O-EPA bioconjugates and is amenable to large scale production. Moreover, it was found that the combination of steps a, b, c and e, thus omitting step d, is already sufficient to typically yield O-EPA conjugates with a purity between 85-95%, such as 85-90%. O-EPA conjugates with a purity of at least 85% may for instance be suitable for the production of veterinary therapeutics such as veterinary vaccines, or for other purposes e.g. for research or diagnostics. Combination of steps a, b and c, thus omitting steps d and e, typically yields O-EPA conjugates with a purity of about 60-80% as measured by SE-HPLC.

In a preferred embodiment, the O-EPA conjugate is in a first step (step a) provided as a filtered periplasmic fraction of prokaryotic host cells comprising the O-EPA conjugate and in a second step (step b) [first chromatography step] an optionally adjusted load of the filtered periplasmic fraction is subjected to a first anion exchange chromatography (AEX 1) step to obtain a first AEX eluate (AEX1).

In a third step (step c) [second chromatography step], an adjusted load of the AEX1 eluate obtained in step b) is subjected to a hydroxyapatite (HA) chromatography step to obtain a HA eluate. In a preferred embodiment the HA chromatography step is a ceramic hydroxyapatite (cHA) chromatography step. Thus, the obtained HA eluate is preferably a cHA eluate.

In a fourth step (step d) [third chromatography step], an adjusted load of the HA or cHA eluate obtained in step c) is subjected to a hydrophobic interaction chromatography (HIC) step to obtain a HIC eluate.

In a fifth step (step e) [fourth chromatography step], an adjusted load of the HIC eluate obtained in step d) is subjected to a second anion exchange chromatography (AEX2) step to obtain a second AEX eluate as product. Including AEX2 as a fourth chromatography step is preferred as this ensures reproducible production of O-EPA bioconjugates with high purity that are suitable for administration in a subject in need thereof.

Again, in each of the second, third, fourth and fifth step, i.e. in each chromatography step, conditions are typically first adjusted to allow binding of the O-EPA conjugate to a chromatography medium and subsequently adjusted to allow elution of the O-EPA conjugate from said medium (“bind-elute mode”).

When following the above protocol, in each of the second, third, fourth and fifth step, i.e. in each of the chromatography steps, the relative amount of O-EPA conjugate versus total protein is higher in the eluate than in the load.

This yields an O-EPA conjugate typically with a purity ≥93%, e.g. 93%, 94%, 95%, 96%, 97%, 98%, 99%, 99.5%, 99.7%, 99.9% or 100% as measured by SE-HPLC. Typically, the purity of the O-EPA conjugate is ≥98%, e.g. 98%, 99%, 99.5%, 99.7% 99.9% or 100% as measured by SE-HPLC, especially when using the process comprising four chromatography steps as described above. A purity of at least 95% or higher, e.g. at least 98%, is advantageous for components of pharmaceutical products that are intended for administration to humans, such as multivalent bioconjugate vaccines comprising multiple O-EPA conjugates.

In certain embodiments, including AEX2 step [fourth chromatography step] or HIC step [third chromatography step] is not required to meet the purity requirements of certain applications, for example for veterinary applications, research or diagnostics. Thus, in certain embodiments, including AEX2 [fourth chromatography step] is optional. In certain other embodiments, including HIC step [third chromatography step] is optional.

Thus, in one embodiment, the O-EPA conjugate is in a first step (step a) provided as a filtered periplasmic fraction of prokaryotic host cells (FPF) comprising the O-EPA conjugate.

In a second step (step b) an optionally adjusted load of the filtered periplasmic fraction is subjected to a first anion exchange chromatography (AEX 1) step to obtain a first AEX eluate (AEX1).

In a third step (step c), an adjusted load of the AEX1 eluate obtained in step b) is subjected to a hydroxyapatite chromatography (HA) step to obtain a HA eluate. The HA chromatography step is preferably a ceramic hydroxyapatite (cHA) chromatography step. Thus the obtained HA eluate is preferably a cHA eluate.

In a fourth step (step d), an adjusted load of the HA or cHA eluate obtained in step c) is subjected to a hydrophobic interaction chromatography (HIC) step to obtain a HIC eluate as product.

In each of the second, third and fourth step, i.e. in each chromatography step, conditions are typically first adjusted to allow binding of the O-EPA conjugate to a chromatography medium and subsequently adjusted to allow elution of the O-EPA conjugate from said medium. This is also typically referred to as “bind-elute mode”.

When following the above protocol, in each of the second, third and fourth step, i.e. in each chromatography step, the relative amount of O-EPA conjugate versus total protein is higher in the eluate than in the load.

This yields an O-EPA conjugate with a purity typically between 85-95%, such as 85-90%, as measured by SE-HPLC.

In another embodiment, the O-EPA conjugate is in a first step (step a) provided as a filtered periplasmic fraction of prokaryotic host cells comprising the O-EPA conjugate and in a second step (step b) an optionally adjusted load of the filtered periplasmic fraction is subjected to a first anion exchange chromatography (AEX 1) step to obtain a first AEX eluate (AEX1).

In a third step (step c), an adjusted load of the AEX1 eluate obtained in step b) is subjected to a hydroxyapatite chromatography (HA) step to obtain a HA eluate. The HA chromatography step is preferably a ceramic hydroxyapatite (cHA) chromatography step. Thus the obtained HA eluate is preferably a cHA eluate.

In a fourth step (step e), an adjusted load of the HA or cHA eluate obtained in step c) is subjected to a second anion exchange chromatography (AEX2) step to obtain a second AEX eluate as product. Accordingly, in this embodiment step d) (HIC) is omitted. An advantage is that this process is shorter, resulting in a cheaper and more convenient process than the process that comprises step d)(HIC); an advantage of the latter process (i.e. including the HIC step) over this shorter process is that the longer process leads to higher purity of the O-EPA product in a more reproducible manner, which is advantageous when preparing pharmaceutical preparations intended for human use.

In each of the second, third, fourth and fifth step, i.e. in each chromatography step, conditions are typically first adjusted to allow binding of the O-EPA conjugate to a chromatography medium and subsequently adjusted to allow elution of the O-EPA conjugate from said medium, i.e. each chromatography step is performed in bind-elute mode.

When following the above protocol, in each of the second, third, fourth and fifth step, i.e. in each chromatography step, the relative amount of O-EPA conjugate versus total protein is higher in the eluate than in the load.

This yields an O-EPA conjugate with a purity typically between 85-95%, such as 85-90%, as measured by SE-HPLC.

This embodiment is preferred over the previous embodiment (i.e. omitting HIC instead of AEX2 is preferred from practical process perspective, although both embodiments result in similar purity of the O-EPA bioconjugate). This embodiment is outlined in example 2.

In optional embodiments, additional filtration steps such as tangential flow filtration, ultrafiltration/diafiltration (a variant of tangential flow filtration), dead-end filtration, and/or sterile filtration, as well as optional concentration and/or dilution and/or buffer change steps for the O-EPA may be added at certain points in the process, but preferably no additional chromatography steps are included, and the process thus preferably does not include more than 4 chromatography steps. As described above, a process according to the invention preferably does not include a size-exclusion chromatography step for purification of the bioconjugate. Of course further purification steps such as chromatography steps could be added to the methods of the invention to get to even higher purity, but this will inevitably lead to lower overall yields, and is thus not desirable because it will likely make the process economically unfeasible for the large scale production of a biopharmaceutical product, while the process of the invention, in particular the most preferred process comprising the four chromatography steps outlined above already leads to the desired purity level for a pharmaceutical product suitable for administration to humans, at a yield that renders the process of the invention economically and practically feasible. It would be possible to explore changing the process to render it even more beneficial, but this will entail additional research of which the outcome is unpredictable.

The process steps outlined above (steps a-e) are explained in more detail below.

Step a: This step serves to provide a filtered periplasmic fraction of prokaryotic host cells comprising the O-EPA conjugate for purification. Providing a filtered periplasmic host cell fraction comprising the O-EPA conjugate is known per se (see e.g. WO 2009/104074, WO 2015/124769 or WO 2020/191082).

To obtain a periplasmic fraction by osmotic shock treatment, cells are first incubated in a buffer with relatively high osmolarity (hypertonic) and then incubated in a buffer with relatively low osmolarity (hypotonic). Such osmotic shock treatment leads to at least partial removal of the cell wall and generation of spheroblasts (i.e. cells, particularly Gram-negative cells, from which the cell wall has been at least partly removed). Thus, the majority of host cell proteins remains in the spheroblasts whereas periplasmic proteins are liberated into the suspension medium.

Typically, E. coli host cells comprising genetic information encoding PglB, EPA and the enzymes for the biosynthesis of the corresponding O-antigen polysaccharide are cultured in a bioreactor, e.g. having a volume of about 100 L-20000 L, e.g. 150 L-5000 L, such as 150 L-1000 L, e.g. 200 L wherein the cells produce the O-EPA. Typically, cells are harvested when they are in the stationary phase, upon cooling the culture to below 20° C., via centrifugation, e.g. via continuous centrifugation e.g. using a disc stack centrifuge. Cells are resuspended in a suitable liquid, e.g. 0.9% NaCl solution or Tris-buffered saline (TBS), and subjected to osmotic shock, preferably using solutions at about 2-15° C., preferably about 6-10° C. Osmotic shock can for instance be performed by addition of sucrose solution (e.g. 60% sucrose, pH 8, 480 mM Tris-HCl, 24 mM EDTA) to the cells to a target concentration of sucrose of about 20-30%, e.g. 25%, incubation of the mixture at about 2-15° C., preferably about 6-10° C. for about 15 minutes to 4 hours, e.g. about 1 hour, while mixing. After the incubation with sucrose the cell/sucrose solution is mixed with a solution of low osmotic value, e.g. by mixing with about 4× the volume of 10 mM Tris-HCL (pH 8.0), at 6-10° C. Mixing can for instance be performed through a static mixer. The O-EPA product is released from the periplasmic space into the supernatant (referred to as periplasmic fraction, PF), and the PF is collected. The material may then be clarified, e.g. by removing cell debris in the PF by separation in a disc stack centrifuge, whereby the supernatant (referred to as centrifuged periplasmic fraction, CPF) is collected. The CPF may be further filtered to remove remaining cell debris, e.g. by filtration through a depth and bioburden reduction filter (e.g. a membrane with a pore size of about 0.2 μm), and the resulting material is collected as filtered periplasmic fraction (FPF). This FPF can subsequently be used in the chromatographic purification process [starting at step b)] of the invention as described herein.

Thus, in a preferred embodiment, step a) further comprises a-1) incubation of a prokaryotic host cell in a bioreactor with a volume of between 100 L and 20000 L, e.g. from 150 L to 5000 L, e.g. 150 L-1000 L, such as 200 L, preferably at a temperature between 34° C. and 36° C., e.g. 35° C., to grow until stationary phase prior to harvest; followed by harvesting the prokaryotic host cells, wherein harvesting preferably comprises a continuous flow centrifugation step to obtain harvested prokaryotic host cells comprising O-EPA conjugate in the periplasm.

Thus, in another preferred embodiment, step a) further comprises a-2), osmotic shock treatment of the host cells to obtain a periplasmic fraction of prokaryotic host cells comprising O-EPA conjugate. It is clear to the skilled person that step a-2 follows step a-1. In certain embodiments, osmotic shock treatment in step a-2) comprises addition of sucrose solution preferably further comprising EDTA to the cells to a target concentration of about 25% sucrose, incubation of the mixture at about 6-10° C. for about 15 minutes to 2 hours while mixing, and subsequently adding a solution having low osmolality (e.g. 10 mM Tris-HCl pH 8) at about 6-10° C. to the cell/sucrose solution to bring down the osmotic value by a factor of at least four (as compared to the composition comprising 25% sucrose), whereby the O-EPA product is released from the periplasm into the supernatant, and collecting the supernatant (periplasmic fraction), and subsequently filtering the periplasmic fraction to obtain a filtered periplasmic fraction.

In a preferred embodiment, the O-polysaccharide is specific to a Gram-negative bacterium selected from the list of Escherichia and Shigella, preferably E. coli.

In additional preferred embodiments, the prokaryotic host cells from which the O-EPA conjugate is obtained comprise genetic information encoding an O-polysaccharide and a recombinant ExoProtein A (EPA) and a metabolic apparatus that carries out N-glycosylation of the EPA with the O-polysaccharide thereby producing the O-EPA conjugate in vivo in the periplasm of the prokaryotic host cells. Details of this aspect are described in more detail above, and in the art, e.g. in WO 2009/104074 and WO 2020/191082.

Step b: This step serves to reduce the processing volume and to reduce process related impurities derived from fermentation procedures, i.e. incubation of the host cell and extraction of a periplasmic fraction comprising the O-EPA conjugate. In particular, impurities that can lead to the degradation of the O-EPA conjugate, such as proteases, peptidases and glycosidases are removed.

Subjecting an optionally adjusted load of the filtered periplasmic fraction to a first anion exchange chromatography (AEX 1) step to obtain a first AEX eluate (AEX1) is known per se. In a preferred embodiment, step (b) further comprises one or more of the following steps (b-1) to (b-2-iii), preferably in the order as indicated:

In one embodiment, this includes (b-1) optionally adjusting a conductivity of a load of the filtered periplasmic fraction suitable for binding to a AEX1 medium.

Adjusting a load of the filtered periplasmic fraction prior to AEX1 is typically not required. Instead, the filtered periplasmic fraction is typically directly subjected to AEX1, unless the conditions, in particular the conductivity, of said filtered periplasmic fraction are not suitable for subjecting to AEX1. A non-limiting example of a suitable AEX1 resin is e.g. a Q-Ceramic HyperD F resin.

In one embodiment, this includes (b-2-i) contacting the optionally adjusted load of the filtered periplasmic fraction with the AEX1 medium, and washing said medium comprising bound O-EPA conjugate with a wash buffer. Typically, said wash buffer has a relatively low salt concentration and a relatively low conductivity. An example of a suitable low salt, low conductivity wash buffer is a buffer at pH 8.5, comprising 10 mM Tris and 50 mM NaCl.

In one embodiment, this includes (b-2-ii) eluting the O-EPA conjugate with an elution buffer, typically comprising a relatively high salt concentration and a relatively high conductivity. An example of a suitable high salt, high conductivity buffer is a buffer at pH 8.5, comprising 10 mM or 50 mM Tris and 1 M NaCl. Typically, elution is performed using a step gradient.

It will be clear for each of these steps that the identity of the AEX1 resin, AEX1 column or membrane or cassette format, the exact buffer components, pH, and/or salt concentration or conductivity as well as the gradient may be varied, as is known to the skilled person based upon the current disclosure.

In one embodiment, this includes (b-2-iii) optionally pooling fractions with an enriched O-EPA conjugate content to obtain an AEX1 eluate. Typically, fractions with an enriched O-EPA conjugate content are identified by performing an SDS-PAGE analysis, or any suitable analysis to determine the amount or relative purity of the desired O-EPA conjugate. Performing SDS-PAGE analysis to thereby identify fractions comprising the intended product, i.e. O-EPA conjugate, is well-known to a person of ordinary skill in the art.

Step c: This step serves to further reduce process related impurities that are present after step b). Typically, after step c), more than 80% of HCPs are depleted compared to the filtered periplasmic fraction provided in step a. In addition, step c) serves to reduce the DNA content and the endotoxin content (in case endotoxins are present; suitable methods for performing endotoxin analysis are known to a person of ordinary skill in the art) compared to a pooled fraction of the AEX1 eluate.

In preferred embodiments, step c, “subjecting an adjusted load of the AEX1 eluate obtained in step (b) to a hydroxyapatite chromatography (HA) step (preferably a ceramic hydroxyapatite chromatography (cHA) step), to obtain a HA or cHA eluate” further comprises one or more of the following steps (c-3) to (c-5-iii), preferably in the order as indicated.

In one embodiment, this includes (c-3), adjusting pH and conductivity of a load of the AEX1 eluate suitable for binding to a HA medium. HA (or cHA) medium that is suitable for this step is commercially available, and a non-limiting example is a CHT Ceramic Hydroxyapatite Type 1 resin. A suitable pH of a load of the AEX1 eluate prior to application on a suitable cHA or HA resin is in particular pH=7.2±0.2. A suitable conductivity of a load of the AEX1 eluate prior to application on a suitable cHA or HA resin is 8.25±0.75 mS/cm. Typically, the pH of the load is adjusted using a suitable buffer, e.g. a buffer comprising 0.5M BisTris HCl and pH 6.0, prior to adjusting the conductivity, e.g. using a buffer comprising 10 mM BisTris, pH 7.0.

In one embodiment, this includes (c-4), optionally performing a particle reduction filtration 1. This step leads to a removal of viable and non-viable particles.

In one embodiment, this includes (c-5-i), contacting the adjusted AEX1 eluate with the HA medium and washing said HA medium comprising the bound O-EPA conjugate with a wash buffer, the wash buffer typically having a relatively low conductivity. An example of a suitable low conductivity wash buffer is a buffer comprising 30 mM BisTris, 3.975 mM K-phosphate, 70 mM NaCl, pH 7.0.

In one embodiment, this includes c-5-ii), eluting the O-EPA conjugate with a salt gradient. An example of a suitable salt gradient is a sodium chloride and potassium phosphate gradient, e.g. a linear gradient of 20-45% of a buffer comprising 30 mM TrisBis, 32 mM K-phosphate, 450 mM NaCl, pH 7.0 (buffer K) in a buffer comprising 30 mM BisTris, 2.5 mM K-phosphate, 50 mM NaCl, pH 7.0 (buffer J) over 2-3, e.g. 2.5, column volumes (CV), followed by a step gradient to 70% buffer K in buffer J for 2-3, e.g. 2.5 CV.

In one embodiment, this includes c-5-iii) optionally pooling of fractions with an enriched O-EPA conjugate content to obtain a HA or cHA eluate.

It will be clear for each of these steps that the identity of the HA or cHA resin, column format, the exact buffer components, pH, and/or salt concentration or conductivity as well as the gradients may be varied, as is known to the skilled person based upon the current disclosure.

Step d: The HIC step serves to further remove HCPs, especially non-glycosylated EPA and a variety of mid-sized HCPs (ca. 40-60 kDa). In preferred embodiments, step d, “subjecting an adjusted load of the HA or cHA eluate obtained in step (c) to a hydrophobic interaction chromatography (HIC) step to obtain a HIC eluate” further comprises one or more of the following steps (d-6) to (d-8-iii), preferably in the order as indicated.

In one embodiment, this includes d-6), adjusting the conductivity of a load of the HA or cHA eluate. Conductivity is adjusted using a loading buffer that is suitable for binding to a HIC medium, the loading buffer typically having a relatively high conductivity. An example of a suitable loading buffer is a buffer comprising 2M K-phosphate at pH 7.0 (buffer Q). A suitable target conductivity in this step is for example a conductivity of 118±2 mS/cm. A suitable HIC medium for this step is a Sartobind Phenyl Jumbo 5 L capsule.

In one embodiment, this includes d-7), optionally performing a particle reduction filtration 2 for the removal of viable and non-viable particles.

In one embodiment, this includes d-8-i), contacting the adjusted HA or cHA eluate with the HIC medium and washing said medium comprising the bound O-EPA conjugate with a suitable buffer, typically a buffer having a relatively high conductivity, e.g. a buffer comprising 2M K-phosphate, pH 7.0, and optionally subsequently performing a second wash step with a buffer having a reduced conductivity (for example a mixture comprising 70% of a buffer comprising 2M K-phosphate, pH 7.0, and 30% WFI water).

In one embodiment, this includes d-8-ii), eluting the O-EPA conjugate using a suitable elution buffer, typically an elution buffer having a relatively low conductivity. Preferably, elution is performed with a step gradient using a suitable elution buffer. An example of a suitable buffer to elute the O-EPA conjugate is a mixture containing 30% of a buffer comprising 2M K-phosphate, pH 7.0, and 70% WFI water.

It will be clear for each of these steps that the identity of the HIC resin, column or membrane or cassette format, the exact buffer components, pH, and/or salt concentration or conductivity as well as the gradient may be varied, as is known to the skilled person based upon the current disclosure.

In one embodiment, this includes d-8-iii), optionally pooling of fractions with an enriched O-EPA conjugate content to obtain a HIC eluate.

Step e: In preferred embodiments, step e, “optionally subjecting an adjusted load of the HIC eluate obtained in (d) to a second anion exchange chromatography (AEX2) step to obtain a second AEX eluate as product” further comprises one or more of the following steps (e-9) to (e-12-iii), preferably in the order as indicated. AEX medium that is suitable for this AEX2 step is commercially available, and a non-limiting example is a Source Q resin.

The AEX2 step serves to further remove process related protein impurities, in particular the E. coli transaldolase (˜37 kDa). Thus, in one aspect the invention provides the use of a polishing step by anion exchange chromatography to reduce the amount of transaldolase in a preparation comprising an O-EPA bioconjugate. In certain embodiments, the O-EPA bioconjugate has undergone previous purification steps including anion exchange, hydroxyapatite, and hydrophobic interaction chromatography steps in that order.

In one embodiment, this includes e-9), performing a tangential flow filtration 1 of a load of the HIC eluate to lower the conductivity and for adaptation of the concentration of the O-EPA conjugate. A suitable concentration of the O-EPA bioconjugate prior to performing the AEX2 step is for example an OD280 of 1.35±0.15. The target concentration may be reached either by diluting the HIC eluate with a suitable buffer, e.g. a buffer with pH 6.0 comprising 10 mM BisTris (buffer T), or by concentrating the HIC eluate by tangential flow filtration. In order to concentrate the HIC eluate by TFF, the volume of the fluid is reduced by allowing permeate flow to occur. Thus, solvent, solutes, and particles smaller than the membrane pore size pass through the membrane, while components larger than the pore size, such as the O-EPA bioconjugate, are retained and thereby concentrated. Such concentration by TFF is followed by diafiltration until a suitable target conductivity is reached. Thus, once the target concentration has been reached, new buffer is added to the feed at the same rate as the permeate flow rate, i.e. the rate at which the feed passes through the membrane. Thereby the HIC eluate is obtained in a suitable buffer and the volume of the concentrated HIC eluate remains constant (buffer exchange). A suitable conductivity is for example 5-6 mS/cm. A suitable buffer for such buffer exchange is for example a buffer at pH 6.0 comprising 10 mM BisTris (buffer T).

In one embodiment, this includes e-10), adjusting a pH and conductivity of a load of the filtered HIC eluate suitable for binding to an AEX2 medium. In some cases, buffer exchange by TFF1 as described above is not sufficient to adjust the pH of the HIC eluate as needed. Thus, the pH of the HIC eluate may be adjusted to a pH of typically 6.5±0.2 using a suitable pH adjustment buffer, e.g. a buffer comprising 0.5M BisTris at pH 6.0 (buffer Y). Further, conductivity of the HIC eluate is adjusted to a target conductivity of typically 8.7±0.3 mS/cm using a suitable conductivity adjustment buffer, e.g. a buffer comprising 10 mM BisTris, 200 mM NaCl at pH 6 (buffer V).

In one embodiment, this includes e-11), optionally performing a particle reduction filtration 3 for removal of viable and non-viable particles.

In one embodiment, this includes e-12-i), contacting the optionally adjusted load of the filtered HIC eluate with the AEX2 medium, and washing said medium comprising bound O-EPA conjugate optionally at least twice with a wash buffer. A suitable wash buffer for this step typically has a relatively low salt concentration and low conductivity, and a non-limiting example of a suitable wash buffer is a buffer containing 10 mM BisTris, 50 mM NaCl, pH 6.0 (buffer U). Optionally a second wash step is performed, and a suitable second wash step for example employs a linear gradient of 15-21% of a buffer containing 10 mM BisTris, 200 mM NaCl, pH 6.0 (buffer V) in buffer U.

In one embodiment, this includes e-12-ii), eluting the O-EPA conjugate with an elution buffer. In certain embodiments, the elution is performed in particular by a step gradient followed by a linear gradient of increasing salt concentration. Typically, the elution buffer has a relatively high salt concentration and high conductivity. One non-limiting example of a suitable process for eluting the O-EPA conjugate is by applying 21% buffer V in buffer U for 3 CV (step gradient), followed by a linear gradient of 21-56% buffer V in buffer U over 7-8, e.g. 7.5 CV.

In one embodiment, this includes e-12-iii), optionally pooling of fractions with an enriched O-EPA conjugate content to obtain an AEX2 eluate as product.

In another preferred embodiment, during step (e), the O-EPA conjugate is bound to an AEX2 matrix and eluted by a step gradient followed by a linear gradient of increasing salt concentration as described above, to obtain O-EPA conjugate with a purity of at least 90%, preferably at least 95%, more preferably at least 98%, or at least 99%.

It will be clear for each of these steps that the identity of the AEX2 resin, AEX2 column or membrane or cassette format, the exact buffer components, pH, and/or salt concentration or conductivity, and the gradients may be varied, as is known to the skilled person based upon the current disclosure.

In another preferred embodiment, the production process further comprises an additional step f) wherein a load of the HIC eluate or preferably of the AEX2 eluate is adjusted to a pharmaceutically acceptable buffer and concentration thereby obtaining the purified O-EPA conjugate as a pharmaceutical drug substance.

In preferred embodiments, step f) further comprises one or more of the following steps (f-13) to (f-17), preferably in the order as indicated:

In one embodiment, this includes f-13), optionally adjusting a pH of a load of the AEX2 eluate suitable for tangential flow filtration 2 (TFF2). A suitable pH for TFF2 is e.g. 6.5±0.2 and one example of a suitable buffer is 100 mM Na₂HPO₄ buffer (Buffer W).

In one embodiment, this includes f-14), performing a TFF2 of the adjusted AEX2 eluate to change to the pharmaceutically acceptable buffer and concentration of the O-EPA conjugate. In one embodiment, the pharmaceutically acceptable buffer comprises 6.19 mM KH₂PO₄, 3.81 mM Na₂HPO₄, 5% (w/w) sorbitol, 10 mM methionine, 0.02% (w/w) polysorbate-80, at pH 7.0.

In one embodiment, this includes f-15), performing a bioburden filtration of the purified O-EPA conjugate to obtain the purified O-EPA conjugate drug substance. Such bioburden filtration can for example be performed using a filter with a PES membrane with 0.45+0.2 μm cut-off, for example a Sartopore 2 Capsule Size 9 filter.

In one embodiment, this includes f-16), portioning and freezing of the purified O-EPA conjugate drug substance to thereby obtain a drug substance bulk.

In another preferred embodiment, the production process further comprises an additional step g), wherein several purified O-EPA conjugate drug substances are combined thereby obtaining a multivalent drug product.

In yet another preferred embodiment, the multivalent drug product comprises at least four, five, six, seven, eight, nine, or ten, purified O-EPA conjugates comprising 0-polysaccharides selected from the list comprising E. coli O-serotypes O1A, O2, O4, O6A, O8, O15, O16, O18A, O25B, and O75. In further embodiments, conjugates of different E. coli serotypes (i.e. O-antigen polysaccharides covalently coupled to carrier protein) may be added, e.g. to obtain a multivalent drug product comprising 10-20 conjugates, e.g. O-EPA conjugates. Such conjugates of different E. coli serotypes may also be bioconjugates, and may also have been purified according to processes described herein.

The following examples of the invention are to further illustrate the nature of the invention. It should be understood that the following examples do not limit the invention and the scope of the invention is to be determined by the appended claims.

### Example 1: This is an Example of a Production Process for ExPEC O-EPA Bioconjugates

This is an example of the 4-column purification process referred to herein as “DSP0”.

The process consists of the following steps: (i) AEX1 (Capture AEX chromatography); (ii) pH and conductivity adjustment for cHA chromatography; (iii) particle reduction filtration 1; (iv) cHA chromatography; (v) pH and conductivity adjustment for HIC; (vi) particle reduction filtration 2; (vii) HIC; (viii) TFF1; (ix) pH and conductivity adjustment for AEX2 (polishing step); (x) particle reduction filtration 3; (xi) AEX2 (polishing chromatography); (xii) pH adjustment for TFF2; (xiii) TFF2; (xiv) bioburden filtration.

Buffers, including their composition, referred to within this example are listed in table 2 below.

Step a: A filtered periplasmic fraction comprising the O25B-EPA bioconjugate served as the starting material for the purification process outlined below. Production of O-EPA bioconjugates has been previously described (see e.g. WO2009/104074, WO2015/124769 and WO2020/191082) and was performed in analogy to these protocols. Specifically, the periplasmic fraction of E. coli host cells comprising an O25B-EPA bioconjugate was obtained by osmotic shock treatment after incubation in a 200 L bioreactor (fermenter) as previously described [WO2020191082]. A filtered periplasmic fraction was obtained, essentially as follows. Incubation in a 200 L bioreactor corresponds to 165 L harvest equivalent (HE; LHE).

In more detail:

a-1: As a harvest step, 165 L culture broth is cooled down to below 20° C., before the harvest by a disc stack centrifuge (DSC) is started, using a separator with a constant flow (e.g. about 160 L/h). The biomass is collected and the centrate (supernatant) is discarded.

a-2: The following step is the osmotic shock, which is performed using solutions at about 6-10° C. The cell wet weight (CWW) for the concentrated harvest is determined and based on the CWW the cell suspension is diluted to a target CWW of for instance about 390 g/L with ⅓ TBS (pH 7.4), and the total volume is determined. 60% sucrose solution is added to the diluted harvest to a target concentration of 25% and incubated at 6-10° C. for 1 h with the cells, while mixing the solution. After the incubation with sucrose the cell/sucrose solution is mixed in-line with 4× the volume of 10 mM Tris-HCL (pH 8.0), at 6-10° C. Mixing is performed through a static mixer. The product is released from the periplasmic space into the supernatant (called periplasmic fraction, PF), and the PF is collected.

Subsequently the material is clarified: the cell debris in the PF is removed by separation in a disc stack centrifuge. A constant flow is applied and based on the centrate turbidity the flow is adjusted. The supernatant is collected, and is referred to as centrifuged periplasmic fraction (CPF).

The CPF still contains cell debris and is filtered through a depth and bioburden reduction filter, and collected as filtered periplasmic fraction (FPF). The FPF was used for further purification of the O-EPA starting with the AEX1 step (step b), as described further below.

### Step b: Capture AEX1 Chromatography

b-1: Adjusting the conductivity of filtered fermenter harvest was not required.

b-2-i: The periplasmic fraction of osmotically shocked and filtered fermenter harvest was loaded in bind-elute mode on a Q Ceramic HyperD F resin for a first Anion Exchange Chromatography (AEX1; 20 cm bed height, 0.34 L resin/L harvest equivalent). After equilibration of the column with the low salt buffer A, the filtered periplasmic fraction comprising the O-EPA bioconjugate was loaded onto the column. The column was then washed with 3 column volumes (CV) of buffer A.

b-2-ii: Elution was achieved using buffer BV2 in a step gradient. The eluate was fractionated in 4 fractions of 0.24 CV, wherein collection of eluate fractions started when the UV-absorption increased with a slope greater than 1.0 AU/min.

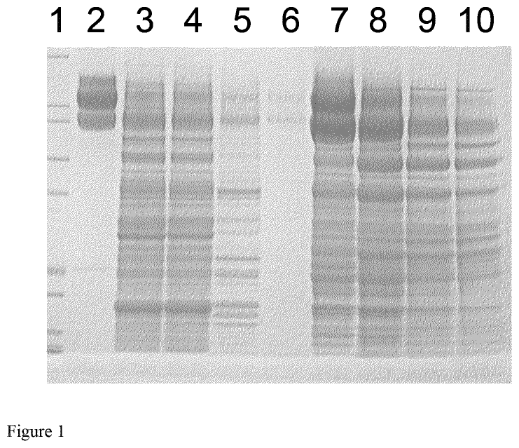

b-2-iii: The individual fractions were analyzed by SDS-PAGE and stained by Coomassie staining (FIG. 1). The “AEX1 pool” (AEX1 eluate) was created by mixing fractions 1 and 2.

### Step c: Hydroxyapatite Chromatography

c-3: The pooled AEX1 fractions were adjusted to a target pH of 7.2±0.2 using Buffer Y and in a subsequent step to a target conductivity of 7.5-9 mS/cm using buffer Z.

c-4: Particle reduction filtration was performed using a PES membrane (Sartopore) with 0.45+0.2 μm cut-off and 0.45 m2/165 LHE filter area.

c-5-i: Ceramic hydroxyapatite chromatography (cHA, 20 cm bed height, 0.34 L resin/L harvest equivalent) was performed in bind-elute mode.

After pre-equilibration with 6 CV Buffer N to adjust pH followed by equilibration with 6 CV Buffer J, the adjusted load of the AEX1 eluate was applied onto the cHA resin. The column was washed using 1 CV of a low conductivity buffer consisting of 5% buffer K in buffer J.

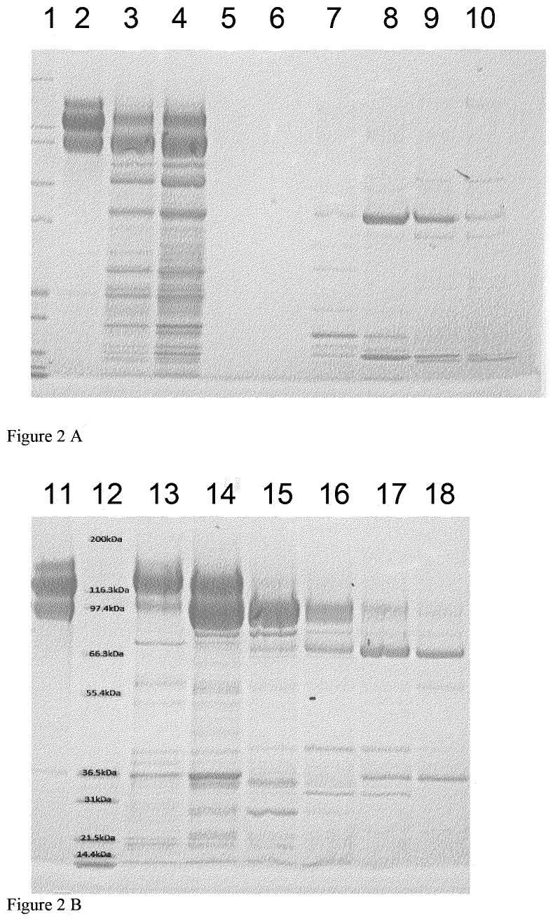

c-5-ii: Selective elution of the product is achieved using a salt gradient, i.e. a sodium chloride and potassium phosphate gradient. At first a linear gradient from 20-45% Buffer K in Buffer J over 2.5 CV was applied, followed by a step gradient to 70% Buffer K in Buffer J for 2.5 CV. Collection of 14 fractions of 15.565 L/165 L HE was started after 1.9 CV of the linear gradient had been applied. C-5-c-5-iii: Again, individual fractions were analyzed by SDS-PAGE (FIG. 2). Selection of fractions for creating a “cHA pool” was based on the intensity of the band corresponding to the O-EPA bioconjugate relative to impurities, i.e. the first fraction selected for pooling contains a strong product band together with only weak to medium-intensity impurity bands and the end of the pooling fractions is marked by fading product bands, often together with a free-EPA band of increasing intensity. The pooled cHA eluate is then created by mixing the selected fractions. In this case, fractions 7-10 were pooled (see FIG. 2).

### Step (d): HIC Chromatography

d-6: To the pooled cHA eluate was added conductivity adjustment buffer (buffer Q) until a target conductivity of 118±2 mS/cm was reached.

d-7: Particle reduction filtration was performed using a PES membrane (Sartopore) with 0.45+0.2 μm cut-off and 0.45 m2/165 LHE filter area.

d-8-i: HIC was performed in bind-elute mode. The HIC capsule (Sartobind Phenyl Jumbo 5 L, 0.8 cm bed height) was equilibrated with 3 CV Buffer Q and the adjusted load of the cHA eluate was applied. Then, the HIC capsule was washed with 1 CV Buffer Q, followed by 4 CV of a buffer mixture containing 70% Buffer Q and 30% Buffer R.

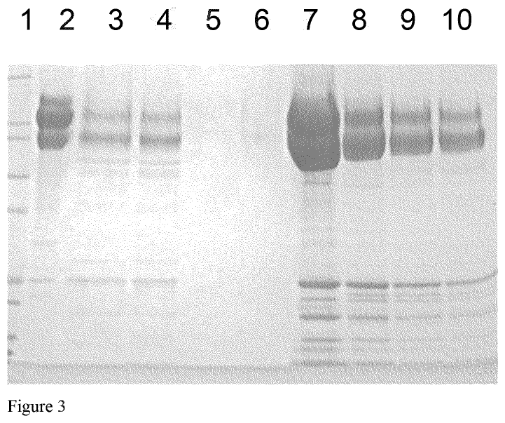

d-8-ii: The O-EPA bioconjugate was eluted by applying 4 CV of a buffer mixture containing 30% Buffer Q and 70% Buffer R. The eluate was fractionated in 4 fractions of 0.8 CV. Collection of eluate fractions started when the UV-absorption increased with a slope greater than 0.1 AU/min. d-8-iii: The individual fractions were analyzed by SDS-PAGE and stained by Coomassie staining (FIG. 3). The “HIC pool” was created by mixing fractions 1-3.

### Step e: Polishing AEX2 Chromatography

e-9: A tangential flow filtration (TFF1) is conducted in order to condition the HIC eluate for the polishing step, i.e. AEX2. The HIC eluate is adjusted (diluted or concentrated) by TFF (mPES KrosFlo Filter Module Q, 10 kDa, 1.25 m²/165 LHE, trans membrane pressure ca. 0.8 bar) with Buffer T to an OD280 of 1.35±0.15 and diafiltered with buffer T to reach the target conductivity of 5-6 mS/cm.

e-10: If pH of the HIC eluate needs additional adjustment prior to the subsequent chromatography step, pH is decreased to a target pH of 6.5±0.2 using buffer Y. In a further step, Buffer V was used to adjust the conductivity to 8.7±0.3 mS/cm.

e-11: The adjusted HIC eluate was then filtered using a Sartopore 2 Capsule Size 0 with a PES membrane with 0.45±0.2 μm cut-off and 0.45 m² filter area.

e-12-i: The polishing AEX chromatography (AEX2) was performed in bind-elute mode. The column (Source 15Q, 20 cm bed height, 6.3 L/165 LHE) was equilibrated using 3 CV Buffer U. The adjusted HIC eluate was applied on the column and a wash step was performed using 1.5 CV Buffer U. In a second wash, a linear gradient of 15 21% Buffer V in Buffer U was applied.

e-12-ii: Elution was performed using first 21% Buffer V in Buffer U for 3 CV (step gradient), then a linear gradient of 21-56% Buffer V in Buffer U over 7.5 CV. Fractionation was started after 1.25 CV of the Elution start and subsequently 20 fractions of 0.4975 CV were collected.

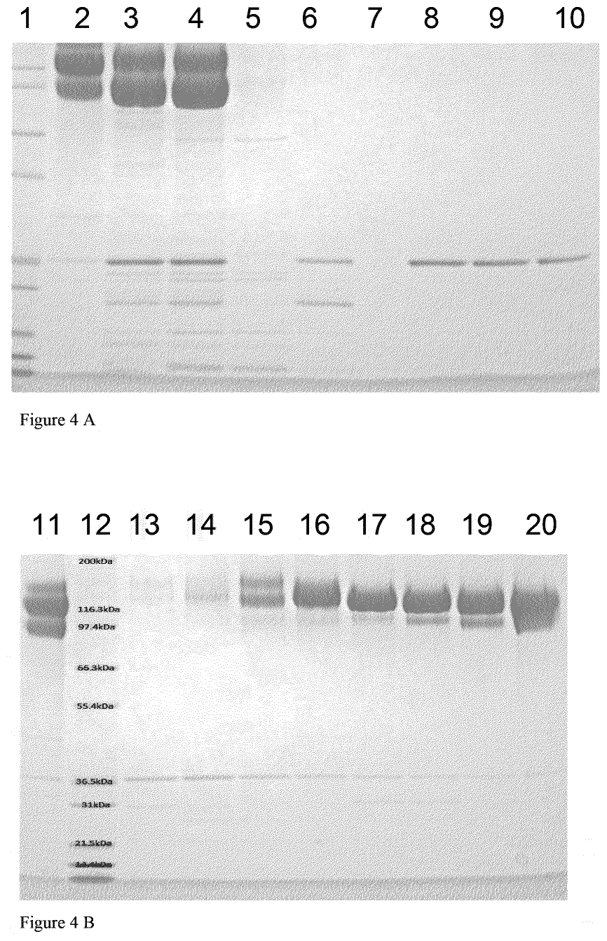

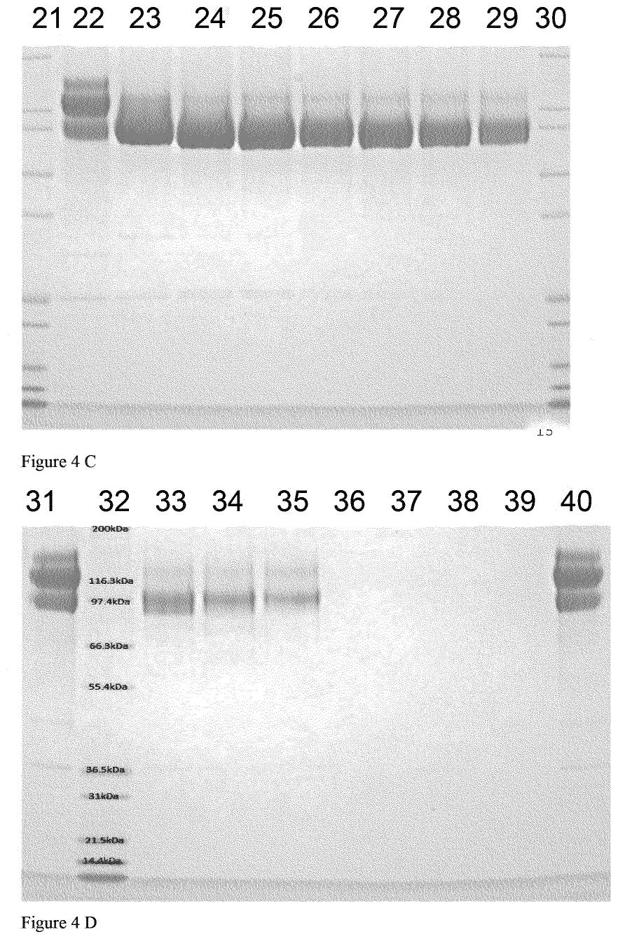

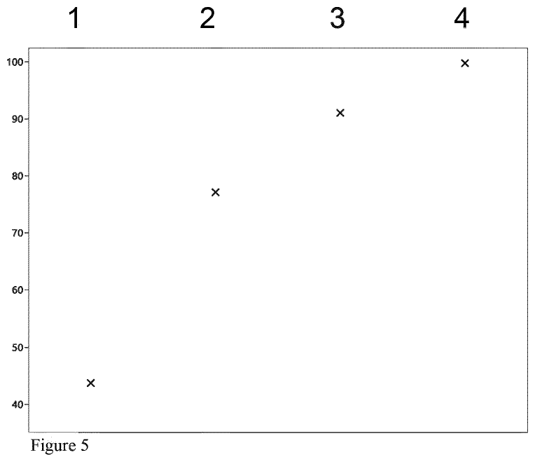

e-12-ii: The individual fractions were analyzed by SDS-PAGE and stained by Coomassie staining (FIG. 4). The AEX2 eluate pool was created by mixing the first fraction with an OD280 of at least 0.04 AU (between fractions 1-20) with the subsequent fractions until fraction 20. In this case, fractions 6-20 were pooled (see FIG. 4). In this example, the O25B-EPA bioconjugate was obtained with a purity of 99.7% as measured by SE-HPLC (FIG. 5).

### Step f: Adjustment to a Pharmaceutically Acceptable Buffer and Concentration

f-13: The pH of the AEX2 eluate was adjusted to a target pH of 6.5±0.2 using Buffer W.

f-14: A second TFF (mPES KrosFlo Filter Module Q, 10 kDa, 1.25 m²/165 LHE) was applied to obtain the O-EPA bioconjugate at a concentration of OD280=0.5±0.1 using an excipient buffer comprising 6.19 mM KH₂PO₄, 3.81 mM Na₂HPO₄, 5% (w/w) sorbitol, 10 mM methionine, 0.02% (w/w) polysorbate-80, at pH 7.0 (Buffer XV2). The adjusted AEX2 eluate was then diafiltered with the excipient buffer over 5-6 diafiltration volumes, and then concentrated to a target concentration of OD280=1.40±0.15. Then, polysorbate-80 (Tween-80) was added to the excipient buffer at a concentration of 0.02% (w/w) to obtain the O25B-EPA conjugate in a pharmaceutically acceptable buffer (Tween-adjusted Buffer XV2) containing 6.19 mM KH₂PO₄, 3.81 mM Na₂HPO₄, 5% (w/w) sorbitol, 10 mM methionine, 0.02% (w/w) polysorbate-80, at pH 7.0 (see e.g. WO 2018/077853).

f-15: A bioburden filtration was applied using a Sartopore 2 Capsule Size 9 with a PES membrane (0.45+0.2 μm cut-off and 0.2 m² filter area).

f-16: The resulting product (drug substance) was filled into bottles (drug substance bulk) and frozen at −70° C.+/−10° C. in a primary freezer. After 72h DS bottles were removed from the primary freezer and directly placed into a final storage freezer at −70° C.+/−10° C. without allowing any thawing of the DS in between.

In this example, the O25B-EPA bioconjugate was obtained with a purity of 99.7%, and a yield of 27 mg PS/L HE, corresponding to an estimated overall yield of about 28% (relative to O-EPA conjugate present in the FPF, of step a-2 above).

A broad range of bioconjugates were purified using the same process as described above. In particular, O-EPA bioconjugates were purified for E. coli serotypes O1A, O2, O4, O6A, O8, O15, O16, O18A, O25B, and O75. The purity for each of these was typically at least 96%, and in most cases purity was 98-100%. The estimated overall yields of the process differed per strain and were inter alia dependent on starting expression levels, and varied between about 5-30%, with a mean of about 18%. This demonstrates that the DSP0 process of the invention is widely applicable for various different O-EPA conjugates, and is suitable for economic large scale production of any O-EPA conjugate to very high purity, which is sufficient for administration of the O-EPA conjugate to humans.

### Step g: Combination of Purified O-EPA Bioconjugate Drug Substances to Obtain a Multivalent Drug Product

The purified drug substances of O-EPA bioconjugates from E. coli O-serotypes O1A, O2, O4, O6A, O8, O15, O16, O18A, O25B, and O75 were mixed in equal quantities (by weight of the polysaccharide for each conjugate) to obtain a 10-valent drug product (see e.g. WO2020/191082), which was suitable for use in humans.

### Example 2: This is an Example of a 3-Column Production Process for ExPEC O-EPA Bioconjugates

In this example, the O-EPA bioconjugate was produced as described in example 1 but step d), i.e. the HIC chromatography step was omitted. In this example, the O25B-EPA bioconjugate was obtained with a purity of 88.3% as measured by SE-HPLC. Thus, a 3-column process omitting the HIC chromatography step results in similar yield and purity as a 3-column process omitting the final AEX2 step (see FIG. 5)

TABLE 2
Buffers

| Name | Composition |
|---|---|
| Buffer A | 10 mM Tris, 50 mM NaCl, pH 8.5 |
| Buffer BV2 | 50 mM Tris, 1M NaCl, pH 8.5 |
| Buffer J | 30 mM BisTris, 2.5 mM K-phosphate, 50 mM NaCl, pH 7.0 |
| Buffer K | 30 mM BisTris, 32 mM K-phosphate, 450 mM NaCl, pH 7.0 |
| Buffer 5% K in J | 30 mM BisTris, 3.975 mM K-phosphate, 70 mM NaCl, pH 7.0 |
| Buffer N | 100 mM BisTris, 2.5 mM K-phosphate, 50 mM NaCl, pH 7.2 |
| Buffer Q | 2M K-phosphate, pH 7.0 |
| Buffer R | WFI water |
| Buffer T | 10 mM BisTris, pH 6.0 |
| Buffer U | 10 mM BisTris, 50 mM NaCl, pH 6.0 |
| Buffer V | 10 mM BisTris, 200 mM NaCl, pH 6.0 |
| Buffer W | 100 mM Na2HPO4 |
| Buffer XV2 | 6.19 mM KH2PO4, 3.81 mM Na2HPO4, 5% (w/w) sorbitol, |
| | 10 mM methionine, 0.02% (w/w) polysorbate-80, pH 7.0 |
| Buffer Y | 0.5M BisTris, pH 6.0 |
| Buffer Z | 10 mM BisTris, pH 7.0 |
| Culture medium | 330 mM Glycerol, 10 g Yeast extract, 20 g Tryptone, 34 mM |
| | K₂HPO₄, 22 mM KH₂PO₄, 38 mM (NH₄)₂SO₄, 2 mM |
| | MgSO₄•7 H₂O and 5 mM citric acid |
| Suspension buffer | 25% Sucrose, 100 mM EDTA 200 mM Tris HCl pH 8.5 |
| Tween-adjusted | 6.19 mM KH₂PO₄, 3.81 mM Na₂HPO₄, 5% (w/w) sorbitol, 10 |
| Buffer XV2 | mM methionine, 0.02% (w/w) polysorbate-80, at pH 7.0 |

(EPA carrier protein including 4 N-linked
glycosylation consensus sequences)
SEQ ID NO: 1
G
SGGGDQNATG SGGGKLAEEA FDLWNECAKA CVLDLKDGVR
SSRMSVDPAI ADTNGQGVLH YSMVLEGGND ALKLAIDNAL
SITSDGLTIR LEGGVEPNKP VRYSYTRQAR GSWSLNWLVP
IGHEKPSNIK VFIHELNAGN QLSHMSPIYT IEMGDELLAK
LARDATFFVR AHESNEMQPT LAISHAGVSV VMAQAQPRRE
KRWSEWASGK VLCLLDPLDG VYNYLAQQRC NLDDTWEGKI
YRVLAGNPAK HDLDIKDNNN STPTVISHRL HFPEGGSLAA
LTAHQACHLP LEAFTRHRQP RGWEQLEQCG YPVQRLVALY
LAARLSWNQV DQVIRNALAS PGSGGDLGEA IREQPEQARL
ALTLAAAESE RFVRQGTGND EAGAASADVV SLTCPVAKDQ
NRTKGECAGP ADSGDALLER NYPTGAEFLG DGGDVSFSTR
GTQNWTVERL LQAHRQLEER GYVFVGYHGT FLEAAQSIVF
GGVRARSQDL DAIWRGFYIA GDPALAYGYA QDQEPDARGR
IRNGALLRVY VPRWSLPGFY RTGLTLAAPE AAGEVERLIG
HPLPLRLDAI TGPEEEGGRV TILGWPLAER TVVIPSAIPT
DPRNVGGDLD PSSIPDKEQA ISALPDYASQ PGKPPREDLK
LGSGGGDQNA T
(example signal sequence for EPA
carrier protein)
SEQ ID NO: 2
MKKIWLALAG LVLAFSASA

1. A process for production of a purified O-polysaccharide-ExoProtein A carrier (O-EPA) conjugate,
wherein said production process comprises:
providing the O-EPA conjugate as a bioconjugate obtained from prokaryotic host cells, said prokaryotic host cells being incubated in a bioreactor with a volume of between 100 L and 20000 L, and
a purification of the O-EPA conjugate comprising the steps of:
providing a periplasmic fraction of prokaryotic host cells that express the bioconjugate,
subjecting the periplasmic fraction to a first anion exchange chromatography (AEX1), followed by
a hydroxyapatite chromatography (HA), followed by
a hydrophobic interaction chromatography (HIC) and followed by
a second anion exchange chromatography (AEX2), and
wherein said purification does not comprise a size-exclusion chromatography (SEC) step.

2. The process according to claim 1, wherein said purification does not comprise an additional chromatography step.

3. The process according to claim 1, wherein said prokaryotic host cells are incubated in a bioreactor with a volume of between 150 L and 5000 L.

4. The process according to claim 1, wherein said production process comprises the steps of:
a) providing a filtered periplasmic fraction of prokaryotic host cells comprising the O-EPA conjugate,
b) subjecting an adjusted load of the filtered periplasmic fraction to a first anion exchange chromatography (AEX 1) step to obtain a first AEX eluate (AEX1), and
c) subjecting an adjusted load of the AEX1 eluate obtained in step (b) to a hydroxyapatite chromatography (HA) step to obtain a HA eluate, and
d) subjecting an adjusted load of the HA eluate obtained in step (c) to a hydrophobic interaction chromatography (HIC) step to obtain a HIC eluate, and
e) subjecting an adjusted load of the HIC eluate obtained in step (d) to a second anion exchange chromatography (AEX2) step to obtain a second AEX eluate as product, and
wherein in purification steps b) to e) conditions are first adjusted to allow binding of the O-EPA conjugate to a chromatography medium and subsequently adjusted to allow elution of the O-EPA conjugate from said medium.

5. The process according to claim 1, wherein the O-polysaccharide is specific to a Gram-negative bacterium selected from the group consisting of Escherichia and Shigella.

6. The process according to claim 1, wherein the prokaryotic host cells from which the O-EPA conjugate is obtained comprise genetic information encoding an O-polysaccharide and a recombinant ExoProtein A (EPA) and a metabolic apparatus that carries out N-glycosylation of the EPA with the O-polysaccharide thereby producing the O-EPA conjugate in vivo in the periplasm of the prokaryotic host cells.

7. The process according to claim 4, wherein step b) comprises the sub-steps of:
b-1) adjusting a conductivity of a load of the filtered periplasmic fraction suitable for binding to a AEX1 medium,
b-2-i) contacting the adjusted load of the filtered periplasmic fraction with the AEX1 medium,
b-2-ii) eluting the O-EPA conjugate,
b-2-iii) pooling of fractions with an enriched O-EPA conjugate content to obtain an AEX1 eluate.

8. The process according to claim 4, wherein step c) comprises the sub-steps of:
c-3) adjusting pH and conductivity of a load of the AEX1 eluate suitable for binding to a HA medium,
c-4) performing a particle reduction filtration 1,
c-5-i) contacting the adjusted AEX1 eluate with the HA medium,
c-5-ii) eluting the O-EPA conjugate,
c-5-iii) pooling of fractions with an enriched O-EPA conjugate content to obtain a HA eluate.

9. The process according to claim 4, wherein step d) comprises the sub-steps of:
d-6) adjusting the conductivity of a load of the HA or cHA eluate,
d-7) performing a particle reduction filtration 2,
d-8-i) contacting the adjusted HA or cHA eluate with the HIC medium,
d-8-ii) eluting the O-EPA conjugate,
d-8-iii) pooling of fractions with an enriched O-EPA conjugate content to obtain a HIC eluate.

10. The process according to claim 4, wherein step e) comprises the sub-steps of:
e-9) performing a tangential flow filtration 1 of a load of the HIC eluate to lower the conductivity and for adaptation of the concentration of the O-EPA conjugate,
e-10) adjusting a pH and conductivity of a load of the filtered HIC eluate suitable for binding to a AEX2 medium,
e-11) performing a particle reduction filtration 2,
e-12-i) contacting the adjusted load of the filtered HIC eluate with the AEX2 medium,
e-12-ii) eluting the O-EPA conjugate,
e-12-iii) pooling of fractions with an enriched O-EPA conjugate content to obtain an AEX2 eluate as product.

11. The process according to claim 4, wherein in an additional step f) a load of the AEX2 eluate is adjusted to a pharmaceutically acceptable buffer and concentration thereby obtaining the purified O-EPA conjugate as a pharmaceutical drug substance.

12. The process according to claim 4, wherein in an additional step g) several purified O-EPA conjugate drug substances are combined thereby obtaining a multivalent drug product.

13. The process according to claim 4, wherein during step (e) the O-EPA conjugate is bound to an AEX2 matrix and eluted by a step gradient followed by a linear gradient of increasing salt concentration, to obtain O-EPA conjugate with a purity of at least 90%.

14. The process according to claim 4, wherein step (a) comprises:
a-1) incubation of the prokaryotic host cells at a temperature between 34° C. and 36° C. to grow until stationary phase prior to harvest;
followed by harvesting the prokaryotic host cells,
wherein harvesting comprises a continuous flow centrifugation step to obtain harvested prokaryotic host cells comprising O-EPA conjugate in the periplasm.

15. The process according to claim 4, wherein step (a) comprises:
a-2) osmotic shock treatment of the prokaryotic host cells, to thereby obtain a periplasmic fraction of prokaryotic host cells comprising O-EPA conjugate.

16. (canceled)

17. The process of claim 11, wherein step f) comprises the sub-steps of:
f-13) adjusting a pH of a load of the AEX2 eluate suitable for tangential flow filtration 2 (TFF2),
f-14) performing a TFF2 of the adjusted AEX2 eluate to change to the pharmaceutically acceptable buffer and concentration of the O-EPA conjugate,
f-15) performing a bioburden filtration of the purified O-EPA conjugate to obtain the purified O-EPA conjugate drug substance,
f-16) portioning and freezing of the purified O-EPA conjugate.

18. The process according to claim 12, wherein the multivalent drug product comprises at least four O-polysaccharides selected from the list comprising E. coli O-serotypes O1A, O2, O4, O6A, O8, O15, O16, O18A, O25B, and O75.


SEQUENCE LISTING (2 sequences)

SEQ ID NO: 1 - protein, 652 aa, Artificial Sequence
  EPA carrier protein including 4 N-linked glycosylation consensus sequences
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 2 - protein, 19 aa, Artificial Sequence
  example signal sequence for EPA carrier protein
MKKIWLALAGLVLAFSASA